(19) United States
(12) Patent Application Publication
(10) Pub. No.: US 2007/0272253 A1
(43) Pub. Date: Nov. 29, 2007

(54) Full Coverage Condom

(76) Inventor: Yuxiang Jason Lin, Davis, CA (US)

Correspondence Address: Yuxiang J. Lin, 4106 Alegre way, Davis, CA, 95616, US

(21) Appl. No.: 11/420,182

(22) Filed: May 24, 2006

(51) Int. Cl. A61F 6/04

(52) U.S. Cl. 128/844

(57) ABSTRACT

A male condom provides full coverage around genital area during intercourse effectively against sexually transmitted diseases and pregnancy. The condom is consisting of two parts. The first part is an elongated tubular that has one open end and one closed end. The second part is an elastic protective film with six adhesive tapes connected to the open end of first part and covers scrotum, entire pubic hair, and peripheral area.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

This invention relates to condom, specifically to a condom that provides greater coverage to effectively against sexually transmitted diseases and pregnancy for users.

Male standard condoms are elongated cylindrical sheaths of thin latex or plastic worn on the penis during intercourse. Condom collects semen before, during, and after a man ejaculates. This can keep sperm from a male to a female vagina to prevent pregnancy and some sexual transmitted diseases.

A major problem of this standard sheath-like is only covering the penis of the male. It cannot provide an impermeable barrier for areas of the skin other than the shaft of the penis that come into contact during sexual intercourse. However, some STDs can be transmitted through skin-to-skin contact (specifically skin with damage) such as human papillomavirus (HPV), and condoms offer little protection against these STDs. Pubic lice can be transmitted by direct physical contact with pubic hair. Another disadvantage of the standard condom is that the man must withdraw immediately after ejaculation so that the condom does not fall off when the penis softens.

There have been some attempts to address this problem by covering scrotum. U.S. Pat. No. 5,718,236 to Curcio (February 1998) discloses ergonomic condom covering scrotum. U.S. Pat. No. 6,209,543 to Carol (April 2001) discloses condom providing enhanced protection against sexually transmitted diseases by using scrotal sac. U.S. Pub. No. 20050188995 to McCoy (April 2005) discloses condom cover all by using resilient pouch to cover scrotum. U.S. Pub. No. 20050103349 to Couvreur (May 2005) discloses a male condom with a collarette to cover the base of the penis.

All above prior art patents are all just covering the scrotum or the base of the penis but not the entire scrotum and pubic hair. They are either difficult to apply, easy slipping partly, or completely off during sexual intercourse. It is difficult to pull on a condom over the scrotum and also difficult to keep the condom snugly on the scrotum during sexual intercourse without causing the user the needless discomfort or even pain.

This invention is advancement over prior art patents in two important ways: (1) the construction of elastic protective film in the open end of the standard condom to cove the scrotum, entire pubic hair, and peripheral area; and (2) the introduction of adhesive conveniently to prevent slippage during sexual intercourse.

Further advantage is the man is able to hold the condom after ejaculation during withdrawal of his penis since the protective film remains outside of the vulva or anus all the time. The protective film also prevents sperm flow outside to contact with his partner's skin.

### SUMMARY OF THE INVENTION

In accordance with the present invention a condom comprises two parts. The first part is an elongated tubular that has one closed end and one open end for inserting the penis. The second part is an elastic rectangle protective film with six adhesive tapes connected to the open end of first part to place over the male external genitalia and peripheral area. This provides sexual partners with full coverage against sexually transmitted diseases and pregnancy.

### BRIEF DESCRIPTION OF THE DRAWINGS

FIG. 1 shows a generally view of the condom of the invention.

FIG. 2 shows a side view of the condom of the invention placed over on a user's body.

### DETAILED DESCRIPTION OF THE INVENTION

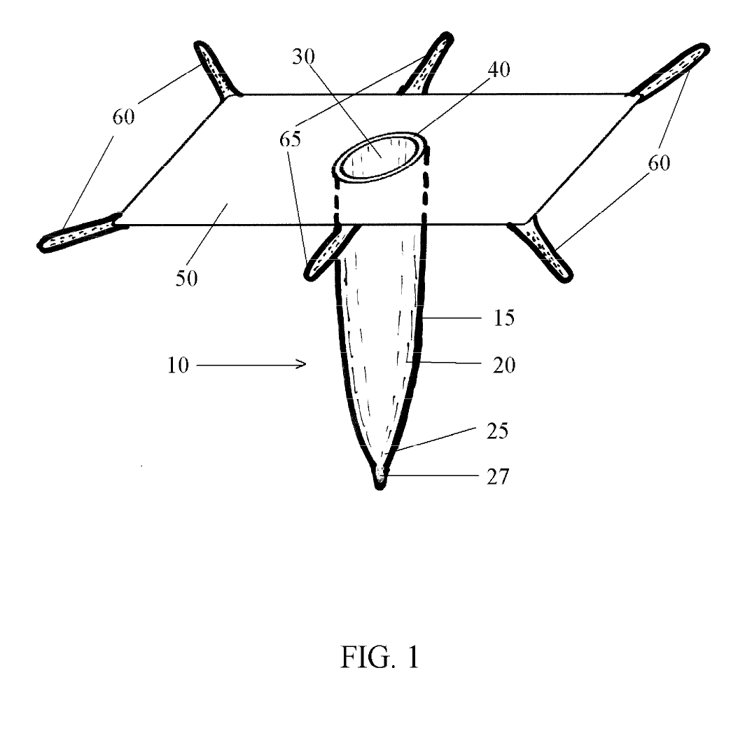

Referring first to FIG. 1, the condom 10 includes a hollow tubular body 20 having a flexible thin-wall 15 with a closed end 25 and an open end 30. The open end 30 comprises a bead 40 that has the same diameter as the tube 20 open end. The bead is made of the same material that has the same diameter as the tube and delimits the protective film with respect to the tube. The closed end 25 includes an optional reservoir tip 27 for containing semen, and the open end 30 for inserting the penis. The rectangle protective film 50 extends in a direction perpendicular to the median axis of the tubular body. The size of this rectangle protective film is about 25×15 centimeters to cover a user's penis, scrotum, entire pubic hair, and peripheral area.

The condom thin-wall 15 can be lubricated inside and/or outside with a lubricant suitable for improving comfort and can be sized in different sizes as needed for different penis size user.

The condom of the invention can be made from standard condom materials such as latex rubber, animal skin, or polyurethane. The wall thickness of 50 can be the same wall thickness of 15, but may be thicker or thinner. The thickness is from 0.01 to 0.25 millimeters.

Four medical adhesive tapes 60 are integrally attached to the four comers and two medical adhesive tapes 65 are integrally attached to the middle of the rectangle protective film 50. The width of the tapes 60 and 65 is from 20 to 40 millimeters. The length of the tapes 60 and 65 is 60 to 100 millimeters. There is a protective layer over the adhesive side of the tape.

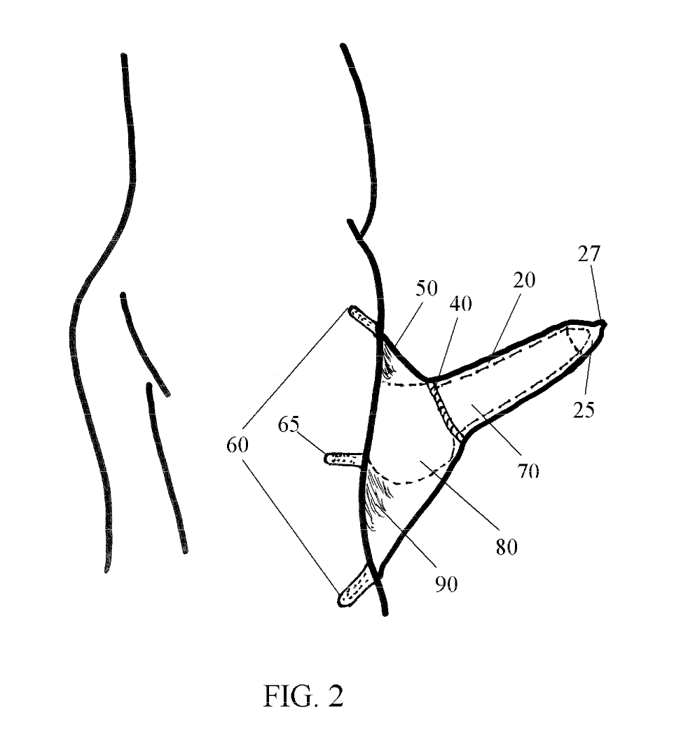

Referring to FIG. 2, the condom of the invention is fitted over the erect male external genitalia, covering penis 70, scrotum 80, pubic hair 90, and peripheral area.

In use, at first the user inserts penis into the condom as standard condom, then unroll the protective film to cover the scrotum, entire pubic hair, and peripheral area, finally peels off the protective layer and press the adhesive firmly to seal.

Unlike a standard condom, it is unnecessary to remove the condom 10 immediately after ejaculation.

Accordingly, the reader will see that the full coverage condom of this invention has simple structure and is easy to use for limiting transmission of bodily fluids during and after intercourse.

Although the above description contains many specificities, these should not be construed as limitations on the scope of the invention, but as merely providing illustrations of some of the presently preferred embodiments of this invention. For example, the protective film can have other shapes, such as trapezoid, oval, etc.

Thus the scope of the invention should be determined by the appended claims and their legal equivalents, rather than by the examples given.

What is claimed is:

1. A male condom configured to cover a user's penis, scrotum, entire pubic hair, and peripheral area, comprising:
a. Flexible thin-walled tubular construction having a closed end and an open end to position over male genitalia.
b. An elastic protective film extending in a direction perpendicular to the median axis of said tubular portion to cover scrotum, pubic hair, and peripheral area.
c. Six medical tapes with adhesive on one side integrally attached to the four corners and the middle of said protective film for conveniently securing to the user's genital peripheral area.

2. The condom as claimed in claim 1, wherein said flexible thin-walled tube and elastic protective film are made from latex rubber, animal skin, or polyurethane.

3. The condom as claimed in claim 1, wherein the open end of the condom comprises an integral bead made of the same material that has the same diameter as said tube, the bead delimiting the protective film with respect to the tube.
